(19) United States
(12) Patent Application Publication
(10) Pub. No.: US 2018/0151255 A1
(43) Pub. Date: May 31, 2018

(54) REMOTE MONITORING OF MEDICAL DEVICES

(71) Applicant: Geneva Healthcare, Inc., Pasadena, CA (US)

(72) Inventors: Manish Wadhwa, San Diego, CA (US); Yuri Sudhakar, Pasadena, CA (US); Jeff Marchese, Scottsdale, AZ (US)

(73) Assignee: Geneva Healthcare, Inc., Pasadena, CA (US)

(21) Appl. No.: 15/884,539

(22) Filed: Jan. 31, 2018

Related U.S. Application Data
(63) Continuation of application No. 15/454,771, filed on Mar. 9, 2017.
(60) Provisional application No. 62/307,255, filed on Mar. 11, 2016.

(51) Int. Cl. G16H 15/00; G16H 10/60; G16H 40/20; G16H 40/63

(52) U.S. Cl. CPC G16H 15/00; G16H 40/63; G16H 40/20; G16H 10/60

(57) ABSTRACT

Methods and systems for calibrating billing cycles for remote monitoring patients and categorizing medical device interrogation data received from remotely monitored patients are disclosed. Interrogation data from a medical device may be received during a current billing schedule. Data from a patient profile may be processed. A determination may be made as to whether a patient's billing schedule corresponds to a first billing schedule or a second billing schedule. A determination may be made as to whether interrogation data from the medical device has been uploaded from a medical device within a current billing cycle preceding the current billing cycle. Upon determining that interrogation data from the medical device has not been uploaded from the medical device within the billing cycle preceding the current billing cycle, the patient's billing schedule may be adjusted such that a date on which interrogation data was received during the current billing schedule corresponds to the last day of the billing cycle preceding the current billing cycle.

### CROSS REFERENCE TO RELATED APPLICATION(S)

This application is a continuation of U.S. application Ser. No. 15/454,771, filed on Mar. 9, 2017, entitled REMOTE MONITORING OF MEDICAL DEVICES, which claims priority to U.S. Provisional Application Ser. No. 62/307,255, filed on Mar. 11, 2016, entitled REMOTE MONITORING OF MEDICAL DEVICES, the disclosures of which are hereby incorporated by reference in their entireties.

### BACKGROUND

Medical device and computer technology have become increasingly sophisticated in recent years. The integration of these technologies has made it possible for medical devices to collect large amounts of information while allowing patients to quickly and conveniently send that information from their home or other remote location to third parties for review. For example, a bedside device may be used to collect vital patient and medical device information from a medical device wirelessly and transmit that information across the Internet or other wireless communication means, to third parties such as health care providers.

Such data collection and communication capabilities have been adopted in a wide range of medical devices. However, with the amount of data that can be generated by and obtained from medical devices, patients, health care professionals, healthcare providers, insurance companies and state and federal agencies confront difficult issues surrounding the amount of useful information that may be provided by these devices.

Third parties who receive patient information derived from medical devices integrated with wireless communication means are faced with ever-increasing problems surrounding this technology. Such problems include issues such as how often the information provided by these devices should be reviewed, what types of information should be reviewed by healthcare professionals and how often patients should provide personal medical information to third parties via these devices for review.

### SUMMARY

In general terms, this disclosure is directed to remote monitoring of medical devices. In one possible configuration and by non-limiting example, the disclosure describes methods and systems for calibrating and categorizing medical device interrogation data received from a medical device. Various aspects are described in this disclosure, which include, but are not limited to, the following aspects.

One aspect is a computer implemented method for calibrating medical device interrogation billing cycles comprising: receiving interrogation data from a medical device during a current billing cycle; processing data from a patient profile; determining whether a patient's billing schedule corresponds to a first billing schedule or a second billing schedule; determining whether interrogation data from the medical device has been uploaded from the medical device within a billing cycle preceding the current billing cycle; and upon determining that interrogation data from the medical device has not been uploaded from the medical device within the billing cycle preceding the current billing cycle, adjusting the patient's billing schedule such that a date on which interrogation data was received during the current billing cycle corresponds to the last day of the billing cycle preceding the current billing cycle.

Another aspect is a computer implemented method for categorizing medical device interrogation data comprising: accessing a patient profile for a patient having a medical device; determining whether a patient's billing schedule corresponds to a first billing schedule or a second billing schedule; determining whether the patient profile has a due report threshold activated, the due report threshold immediately preceding the end of a current billing cycle; determining whether interrogation data from the medical device has been received in a reimbursable period within a current billing schedule; and upon determining that interrogation data from the medical device has not been received during a reimbursable period within the current billing schedule, displaying, via a graphical user interface, an indication that interrogation data from the medical device is due.

A further aspect is a computer implemented method for categorizing medical device interrogation data comprising: accessing a patient profile for a patient having a medical device; determining whether a patient's billing schedule corresponds to a first billing schedule or a second billing schedule; analyzing one or more billing cycles for the patient to determine whether an interrogation was received for each of the one or more billing cycles; and displaying, via a graphical user interface, an indication of whether an interrogation was received for each of the one or more billing cycles.

Yet another aspect is a computer implemented method for determining the billable status of a medical device's interrogation comprising: accessing a patient profile for a patient having an medical device; determining whether a patient's billing schedule corresponds to a first billing schedule or a second billing schedule; analyzing interrogation data for a billing cycle received from the medical device, the interrogation data corresponding to a first interrogation; determining whether the interrogation data falls within a black-out term corresponding to the first billing schedule or the second billing schedule; determining whether interrogation data corresponding to a second interrogation within the billing cycle has been received, the second interrogation preceding the first interrogation; and displaying, via a graphical user interface, an indication of whether the first interrogation is a reimbursable interrogation.

### BRIEF DESCRIPTION OF THE DRAWINGS

FIG. 1 is a schematic diagram illustrating an example care system involving implantable cardiac devices, including an interrogation data management system.

FIG. 2 is a timeline representative of three consecutive 30-day billing cycles, including 10-day black-out terms at the start of each billing cycle.

FIG. 3 is a flow chart illustrating an exemplary method for implementing an interrogation data management system.

FIG. 4 is a screen shot illustrating an example user interface for setting up a remote monitoring billing schedule in a patient profile.

FIG. 5 is a screen shot illustrating an example user interface for creating a patient profile for a patient enrolling in a remote monitoring program.

FIG. 6 is a screen shot illustrating an example user interface demonstrating the contrast between a billable interrogation and a non-billable interim interrogation.

FIG. 7 is a timeline representative of a 30-day billing cycle, including a 10-day black-out term at the start of the billing cycle and a 10-day due report deadline at the end of the billing cycle.

FIG. 8 is a flow chart illustrating an exemplary method for determining whether medical device interrogation data is due to be uploaded by a patient enrolled in a remote monitoring program.

FIG. 9 is a screen shot illustrating an example user interface for determining which patients enrolled in a remote monitoring program are due to upload medical device interrogation data.

FIG. 10 is a timeline representative of a 30-day billing cycle, including a 10-day black-out term at the start of the billing cycle, in which no interrogations have been received.

FIG. 11 is a flow chart illustrating an exemplary method for determining whether an interrogation data upload from a patient's medical device was missed during a billing cycle.

FIG. 12 is a screen shot illustrating an example user interface for determining which patients enrolled in a remote monitoring program did not upload interrogation data from their medical devices during a previous billing cycle.

FIG. 13 illustrates two timelines demonstrating three consecutive 30-day billing cycles implementing automatic calibration of an interrogation received in the third billing cycle.

FIG. 14 is a flow chart illustrating an example method for calibrating a first billing cycle in which an interrogation has been received and a preceding billing cycle in which no billable interrogations were received.

FIG. 15 is a simplified distributed computing network in which various aspects of the present disclosure may be practiced.

FIG. 16 is a block diagram illustrating example physical components of a computing device with which aspects of the disclosure may be practiced.

FIG. 17 is a block diagram illustrating physical components (e.g., hardware) of a computing device 1700 with which aspects of the disclosure may be practiced.

### DETAILED DESCRIPTION

Various embodiments will be described in detail with reference to the drawings, wherein like reference numerals represent like parts and assemblies throughout the several views. Reference to various embodiments does not limit the scope of the claims attached hereto. Additionally, any examples set forth in this specification are not intended to be limiting and merely set forth some of the many possible embodiments for the appended claims.

The various embodiments described above are provided by way of illustration only and should not be construed to limit the claims attached hereto. Those skilled in the art will readily recognize various modifications and changes that may be made without following the example embodiments and applications illustrated and described herein, and without departing from the true spirit and scope of the following claims.

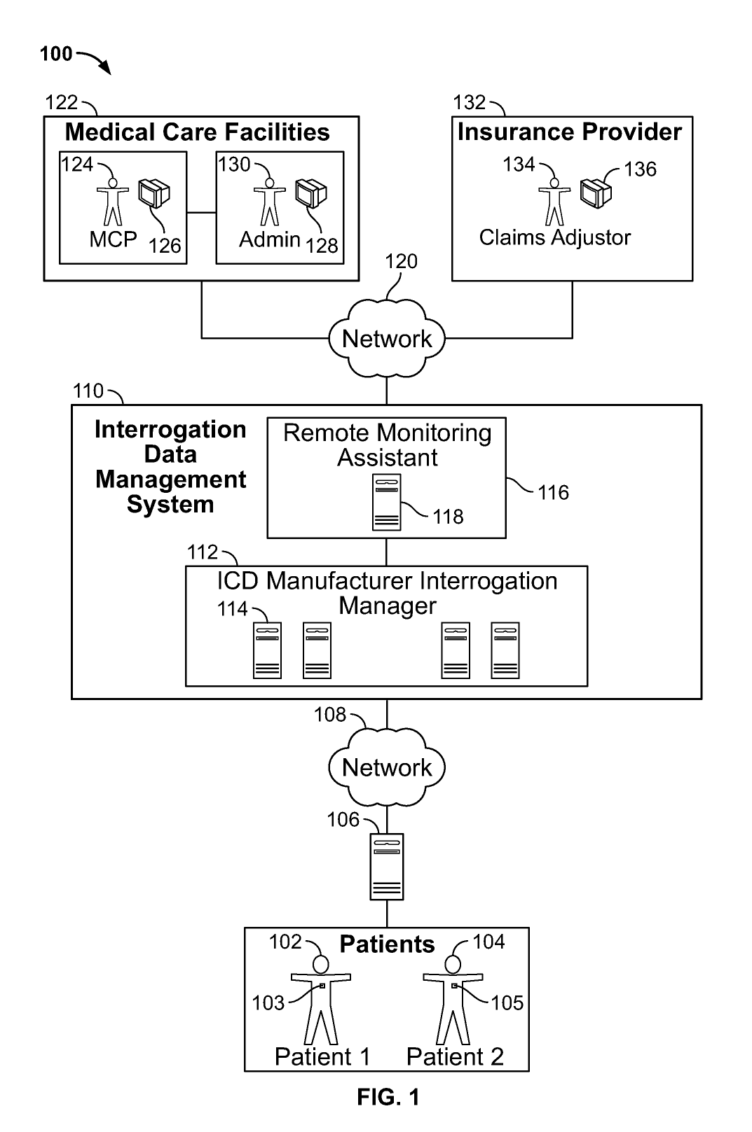

FIG. 1 is a schematic diagram of an example care environment 100 involving medical devices, such as implantable cardiac devices 103 and 105. In this example the care environment 100 includes an interrogation data management system 110. In some embodiments the care environment also includes patients 102, medical care facilities 122, insurance provider 132, and one or more servers 106 in communication with interrogation data management system 110 via data communication network 108.

Patients 102 having implantable cardiac devices 103 and 105 (ICDs) are cared for by the medical care facilities 122 and the medical care professionals MCP 124 associated with those facilities 122. Implantable cardiac devices 103 and 105 typically include devices such as single, dual or multiple lead pacemakers, single, dual or multiple lead defibrillators, cardiovascular monitors and loop recorders, by way of example. Other implantable and non-implantable medical devices may be remotely monitored according to the methods and systems disclosed herein, such as insulin pumps, left ventricular assist devices and wearable cardiac medical devices (e.g., wearable cardioverter-defibrillators).

Medical care facilities 122 typically include one or more administrators 130 responsible for compiling information from the medical care professionals MCP 124 regarding care they have provided to patients 102 and time spent reviewing medical information pertaining to patients 102 (e.g., medical device interrogation), as well as sending invoices for that work to one or more insurance providers 132 and their corresponding claims adjustors 134 for reimbursement.

According to aspects the administrators 130 are employees of the medical care facilities 122. According to other aspects the administrators 130 are not be employees of the medical care facilities 122. For example, the administrators may work for a third-party that is utilized by medical care facilities 122 and the third party may use the remote monitoring assistant 116 to compile and send interrogation data that has been reviewed by one or more medical care professionals MCP 124 to the one or more insurance providers 132 and their corresponding one or more claims adjustors 134 for reimbursement of the services provided by the medical care facilities 122.

The one or more claims adjustors 134 typically review the invoices provided by the one or more administrators 130 and ensure the information provided in the invoices they have been provided with reflect billable actions taken by the medical care facilities 122. For example, the one or more claims adjustors 134 may evaluate the information provided in the invoices and determine whether the actions described therein correspond to reimbursable medical classification or medical coding that is reimbursable under a patient's insurance policy.

According to aspects the one or more claims adjustors 134 may obtain and review an invoice provided by a medical care facility on one or more computing devices 136 and determine whether the actions described in that invoice correspond to a code reflected in a reimbursable medical code set covered by a patient's insurance policy. Code sets the claims adjustors 134 may consult in their review include the Current Procedural Terminology code set, which is a medical code set maintained by the American Medical Association which describes medical, surgical and diagnostic services. Medical code sets such as the Current Procedural Terminology code set are generally designed to communicate uniform information about medical services and procedures among physicians, coders, patients, accreditation organizations and payers for administrative, financial and analytic purposes.

In some cases the implantable cardiac devices 103 and 105 permit remote monitoring, such as by wirelessly communicating interrogation data to a remote monitoring device at the patient's home or work. The transfer of data from the implantable cardiac devices 103 and 105 to another device is often referred to as an interrogation and the data obtained therefrom is often referred to as the interrogation data (i.e., the data obtained may be reviewed, or interrogated, by the medical care professionals MCP 124). Interrogations may also occur when the patients 102 visit medical care facilities 122. For example, when a medical care professional MCP 124 learns that one of the patients 102 has an implantable cardiac device 103 or 105, the medical care professional MCP 124 may order an interrogation of that device.

The medical care professionals MCP 124 are people with medical training, including physicians and nurse practitioners, for example. Some medical care professionals MCP 124 may provide direct care to patients 102, while other medical care professionals MCP 124 may not interact directly with patients 102, but may be involved in other ways, such as reviewing interrogation data, for example. In either case, the medical care professionals MCP 124 can all be said to be caregivers who are providing care to the patients 102 in one form or another. One example of a physician is an electrophysiologist. Electrophysiologists specialize in diagnosing and treating problems with the heart's electrical system. Other physicians can be involved as part of a medical care professional care team, such as an emergency room physician, a primary caregiver, and the like. In some embodiments nurse practitioners assist physicians with certain tasks, such as some of the tasks described herein.

One or more medical care professionals MCP 124 may obtain interrogation data uploaded by a patient and review it on a computing device 126 for prognostic and diagnostic indications related to the patient. The one or more medical care professionals MCP 124 may make a determination based on that review, indicate that that the interrogation data has been reviewed, by for example electronically signing the record via the computing device 126, and attach comments to the reviewed interrogation data, which may be reflected on the patient's electronic medical record.

According to some aspects, one or more medical device technicians may also be involved, as part of the medical care professional team, with the receipt and review of interrogation data. Medical device technicians may provide support to medical care professionals MCP 124 in the way of obtaining interrogation data from third-parties, such as entities running the remote monitoring assistant 116 and the ICD manufacturer interrogation manager 112, fixing issues that may be hindering the generation of readable reports on a computing devices 126, and other technical issues that may hinder a medical care professional from performing an interrogation of a patient's implantable medical device.

Patients 102 may upload interrogation data from their implantable cardiac devices 103 and 105 to the interrogation data management system 110 via the one or more servers 106 and the data communication network 108. The data communication network 108 may include a local area network or a wide area networking environment. When used in a local area networking environment or a wide area network environment (such as the Internet), the interrogation data management system 110 is typically connected to the network 108 through a network interface, such as an Ethernet interface. Other possible embodiments use other communication devices. For example, some embodiments of the interrogation data management system 110 include a modem for communicating across the network 108.

Patients 102 may directly upload interrogation data (e.g., medical device battery voltage and longevity, lead impedance and trends, A-V conduction histograms, and patient cardiac information such as arrhythmia summaries) to the interrogation data management system 110 from their implantable cardiac devices 103 and 105, or via one or more additional computing devices. Such additional computing devices may include bedside monitors, telemetry wands, as well as intermediate servers 106 which may be in communicative contact, via wired telephonic, or wireless communication devices (e.g., cellular, Wi-Fi, Bluetooth, Internet), with the interrogation data management system 110.

The interrogation data may be provided to ICD manufacturer interrogation manager 112 and ICD manufacturer interrogation servers 114. The interrogation data may be further passed along to remote monitoring assistant 116 and remote monitoring assistant servers 118. Alternatively or additionally the interrogation data may be passed directly from the patients 102 to the remote monitoring assistant 116 and remote monitoring assistant servers 118, without first being sent to the ICD manufacturer interrogation manager 112 and the ICD manufacturer interrogation servers 114.

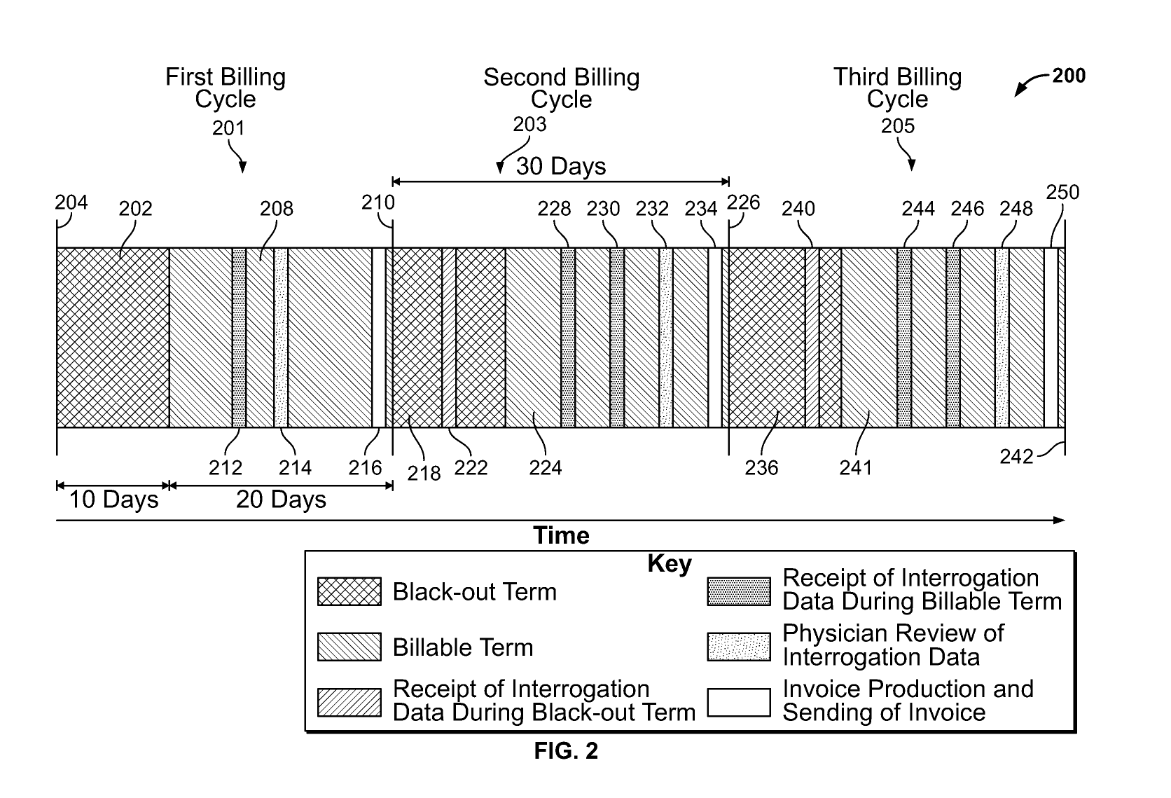

Turning to FIG. 2 a timeline 200 representative of three consecutive billing cycles 201, 203 and 205 is provided. Each of the billing cycles includes a black-out term 202, 218 and 236 corresponding to the first 10 days in a billing cycle in which a review by a medical care professional of uploaded interrogation data during that term is not a billable action. More specifically, each of black-out terms 202, 218 and 236 is a term in a billing cycle that is designated by insurance policies as a term in which a performed action (e.g., physician review of interrogation data uploaded during that term) performed by a medical care professional is not reimbursable.

Each of the billing cycles 201, 203 and 205 also contains a 20-day billable term 208, 224 and 241 immediately following each black-out term 202, 218 and 236. For each billable term 208, 224 and 241 a medical care provider may bill an insurance company for reviewing the first received interrogation data during that billable term. A medical care provider may not bill an insurance company for reviewing any interrogation data that was received after the first uploaded interrogation data during the same billable term.

The 30-day billing cycles and their corresponding 10-day black-out terms depicted in FIG. 2 may also be representative of other billing cycles, such as 90 day billing cycles and corresponding 30-day black-out terms as may be designated by insurance carriers and their corresponding reimbursement policies which may change periodically.

The first billing cycle 201, beginning at 204 and ending at 210, includes billable term 208, reception of interrogation data 212 by an interrogation data management system within billable term 208, medical care professional review 214 of that interrogation data and invoice production and sending of that invoice 216 to one or more insurance providers.

In the first billing cycle 201 reception of interrogation data 212 corresponds to billable interrogation data because it was received during a billable term 208.

The second billing cycle 203, beginning at 210 and ending at 226, includes black-out term 218, reception of interrogation data 222 during that black-out term 218, billable term 224, reception of interrogation data 228 and 230 by an interrogation data management system within billable term 224, medical care professional review 232 of that interrogation data and invoice production and sending of that invoice 234 to one or more insurance providers.

In the second billing cycle 203, reception of interrogation data 222 does not correspond to billable interrogation data because it was received during black-out term 218. Reception of interrogation data 228 does correspond to billable interrogation data because it is the first reception of interrogation data during a billable term 224. Reception of interrogation data 230 does not correspond to billable interrogation data because it is not the first interrogation data received during a billable term 224.

The third billing cycle 205, beginning at 226 and ending at 242, includes black-out term 236, reception of interrogation data 240 during that black-out term 236, billable term 241, reception of interrogation data 244 and 246 by an interrogation data management system within billable term 241, medical care professional review 248 of that interrogation data and invoice production and sending of that invoice 250 to one or more insurance providers.

In the third billing cycle 205, reception of interrogation data 240 does not correspond to billable interrogation data because it was received during black-out term 236. Reception of interrogation data 244 does correspond to billable interrogation data because it is the first reception of interrogation data during a billable term 241. Reception of interrogation data 246 does not correspond to billable interrogation data because it is not the first interrogation data received during a billable term 241.

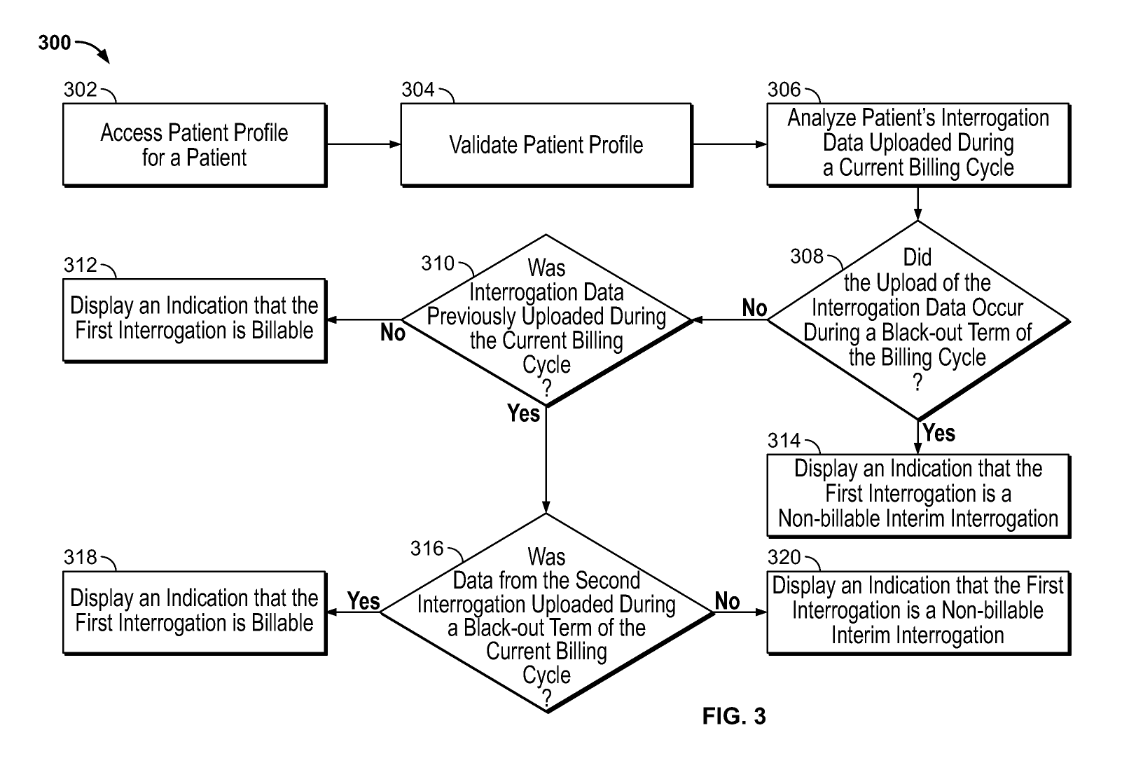

FIG. 3 is a flow chart 300 illustrating an exemplary method for implementing an interrogation data management system according to the current disclosure. The method begins at operation 302 where a patient profile for a patient is accessed. Flow continues to operation 304 where the patient profile is validated and at operation 306 a patient's interrogation data uploaded during a current billing cycle is analyzed.

Validation of the patient profile may entail determining that the patient is enrolled in a remote monitoring program, and subsequently whether the patient is enrolled in a 30-day billing schedule, a 90-day billing schedule, or both a 30-day billing schedule and a 90-day billing schedule.

Moving to operation 308 a determination as to whether the uploaded interrogation data occurred during a black-out term of the current billing cycle is made. When a determination is made that the uploaded interrogation data occurred during a black-out term of the current billing cycle flow continues to operation 314. When a determination is made that the uploaded interrogation did not occur during a black-out term of the current billing cycle flow continues to operation 310.

At operation 314 an indication that the first interrogation is a non-billable interim interrogation is generated and displayed and the method ends.

At operation 310 a determination is made as to whether interrogation data was previously uploaded during the current billing cycle. When a determination is made that that interrogation data was not previously uploaded during the current billing schedule flow moves to operation 312. When a determination is made that interrogation data was previously uploaded during the current billing schedule flow moves to operation 316.

At operation 312 an indication that the first interrogation is a billable interrogation is generated and displayed and the method ends.

At operation 316 a determination is made as to whether data from the second interrogation was uploaded during a black-out term of the current billing cycle. When a determination is made that data from the second interrogation was not uploaded during a black-out term of the current billing cycle flow continues to operation 320. When a determination is made that data from the second interrogation was uploaded during a black-out term of the current billing cycle flow continues to operation 318.

Flowing to 318 an indication that the first interrogation is a billable interrogation is generated and displayed and the method ends.

Flowing to operation 320 an indication that the first interrogation is a non-billable interim interrogation is generated and displayed and the method ends.

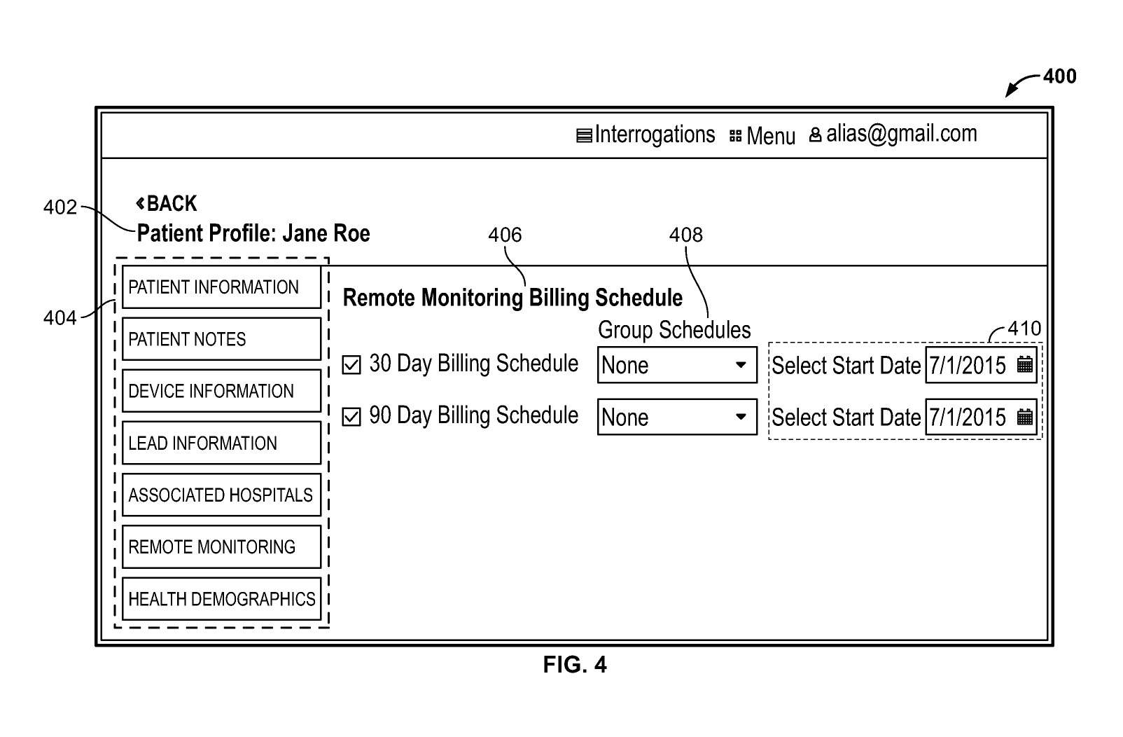

FIG. 4 is a screen shot illustrating an example user interface 400 for setting up a remote monitoring billing schedule in a patient profile 402 for patient Jane Roe. The example user interface 400 contains a selection menu 404 including selectable options for patient information, patient notes, device information, lead information, associated hospitals, remote monitoring and health demographics.

The user interface 400 displays the remote monitoring option which further includes a remote monitoring billing schedule option 406, a group schedules option 408 and a select start date option 410. The remote monitoring billing schedule option 406 allows a user to select 30-day and 90-day billing schedules for a patient, which will determine the functionality of features in the remote monitoring application such as auto-calibration of billing cycles, what dates within a billing cycle are black-out terms or potential billable interrogation dates, as well as when missed and due reports should be generated for the patient.

The group schedules option 408 allows a user to place the patient on a collective billing cycle with one or more other patients such that the start of a billing cycle for each patient in the collective billing cycle corresponds to the same date. The select start date option 410 allows a user to choose a date on which the patient will begin a 30-day or 90-day billing cycle. As reflected in the user interface 400 patient Jane Roe would be placed on both a 30-day and 90-day individual billing schedule, both of which would begin on Jul. 1, 2015.

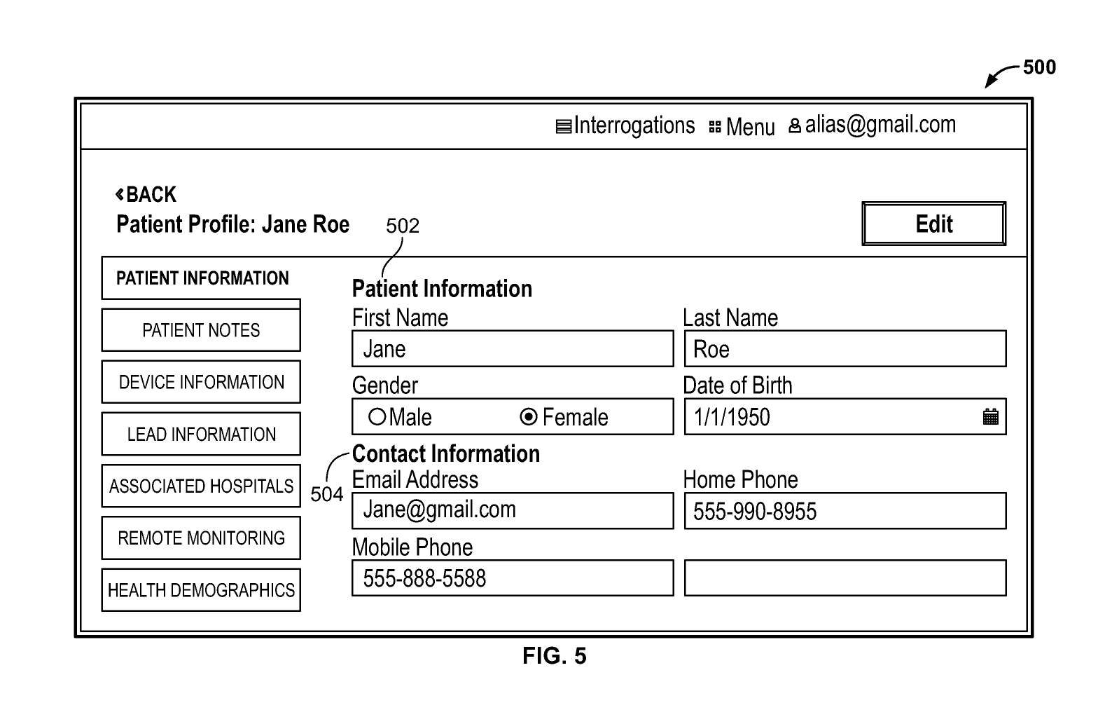

FIG. 5 is a screen shot illustrating an example user interface 500 for inputting personalized patient information 502 and contact information 504 in a patient profile for a patient enrolled or enrolling in a remote monitoring program. Personalized patient information 502 may include input categories for the patient including first and last name, gender and date of birth. Contact information 504 may include categories such as email address and home and mobile phone numbers. Contact information 504 may be utilized by a medical care facility administrator or third-party remote monitoring technician who utilizes a remote monitoring assistant application in an interrogation data management system. For example, a medical care facility administrator or third-party remote monitoring technician may use a patient's contact information 504 to notify the patient if and when the patient is due to upload interrogation data during a billing cycle.

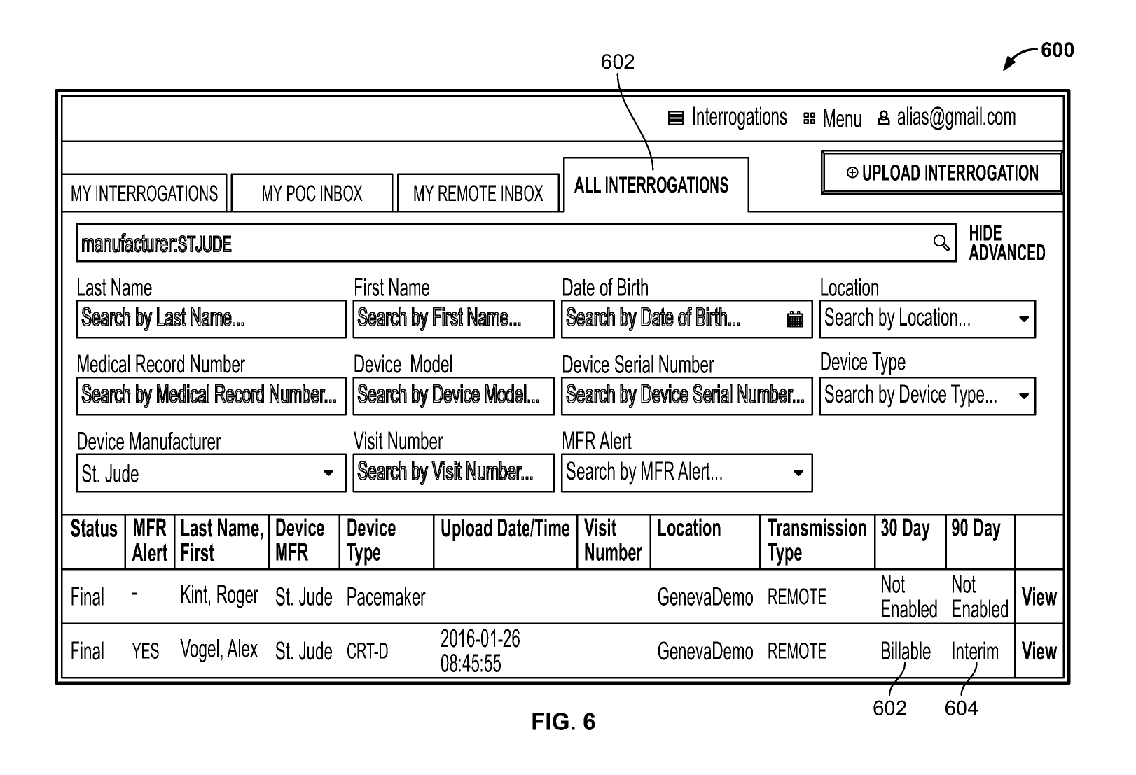

FIG. 6 is a screen shot illustrating an example user interface 600 demonstrating the contrast between a billable interrogation 602 and a non-billable interim interrogation 604. According to examples a user may select an all interrogations option 606 in a remote monitoring assistant application which will generate a list of all patients enrolled in a remote monitoring program for a medical care facility. According to examples the list may contain information for each of those patients pertaining to their remote monitoring schedule including patient name, device manufacturer, device type, date on which interrogation data was last uploaded and the billable status of the last uploaded interrogation data. In addition to billable and interim status indicators the user interface may generate a status of NOT ENABLED for a last uploaded interrogation if 30-day and 90-day remote monitoring schedules have not been activated for a patient.

According to aspects the user interface 600 may provide advanced search criteria for sorting interrogations provided by the remote monitoring application. One or more advanced search criteria may be input into the remote monitoring application to filter the interrogation results that are displayed on the user interface 600, including first and last name, date of birth, location, medical record number, device model, device serial number, device type, device manufacturer, visit number and manufacturer alert.

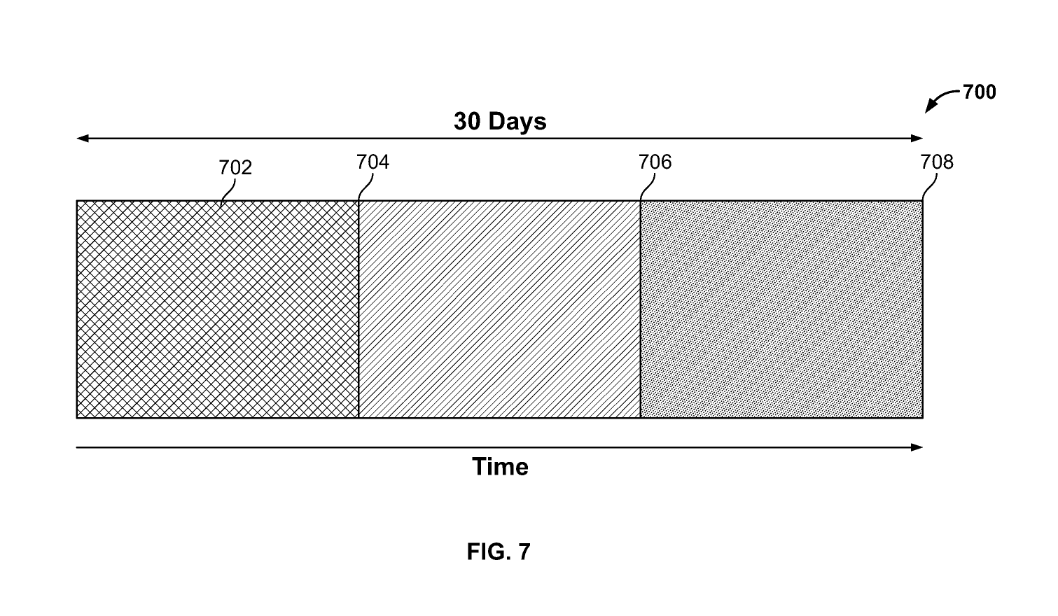

FIG. 7 is a block diagram illustrating an example timeline 700 of a billing cycle which may be utilized for issuing and sending due reports. In this example, the timeline 700 has a single 30-day billing cycle, including 10-day black-out term 702 which corresponds to days 1-10 at the start of the 30-day billing cycle, a due report threshold 706 activated 10 days prior to the end of the billing cycle, and a 20-day billable term running from the end 704 of the 10-day black-out term through the last day 708 of the billing cycle (days 11-30). The 30-day billing cycle and its corresponding 10-day black-out term 702 and the due report threshold 706 depicted in timeline 700 may also be representative of other billing cycles, such as a 90-day billing cycle having a 30-day black-out term and a due report threshold activated 30 days prior to the end of the billing cycle as may be designated by one or more insurance carriers for remote monitoring of a particular medical device or a patient medical condition that is being remotely monitored.

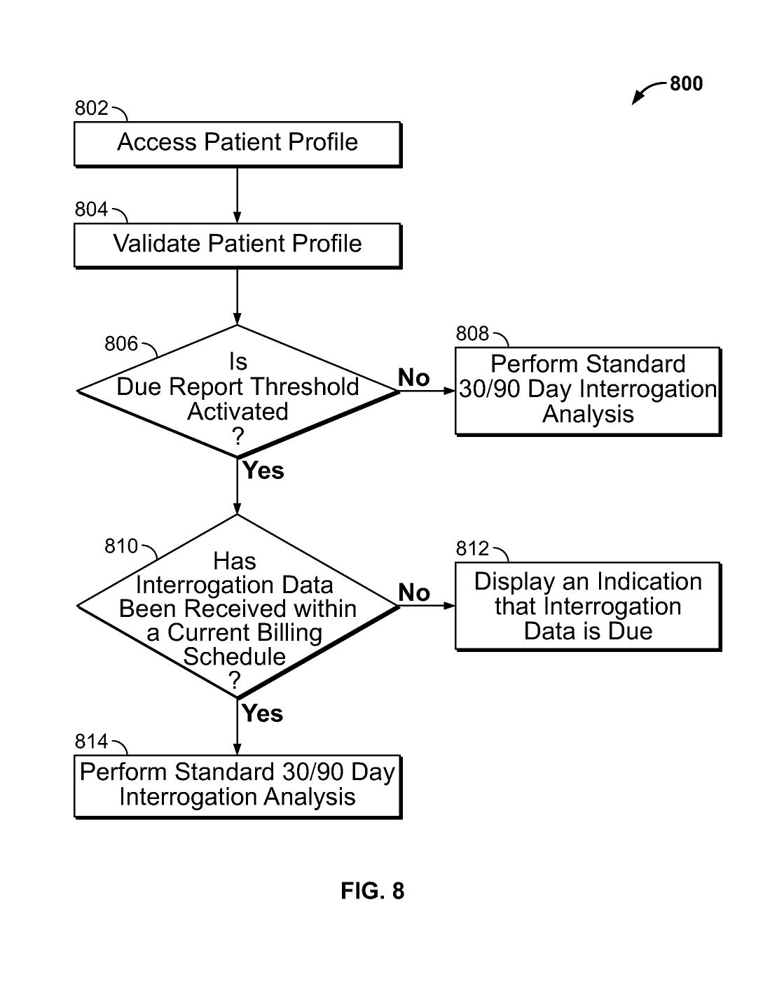

FIG. 8 is a flow chart 800 illustrating an exemplary method for determining whether medical device interrogation data is due to be uploaded to an interrogation data management system by a patient enrolled in a remote monitoring program. The method begins at operation 802 where a patient profile for a patient is accessed and flow continues to operation 804 where the patient profile is validated.

Validation of the patient profile may entail determining that the patient is enrolled in a remote monitoring program, and subsequently whether the patient is enrolled in a 30-day billing schedule, a 90-day billing schedule, or both a 30-day billing schedule and a 90-day billing schedule.

Moving to operation 806 a determination is made as to whether a due report threshold has been activated for a patient. When a determination is made that a due report threshold has not been activated for the patient flow continues to operation 808 where a standard 30-day or 90-day interrogation analysis is performed depending on whether the patient is enrolled in a 30-day or 90-day billing schedule and the method ends. When a determination is made that a due report threshold has been activated for the patient flow continues to operation 810.

At operation 810 a determination is made as to whether interrogation data for the patient has been uploaded and received by an interrogation data management system during the current billing cycle. When a determination is made that interrogation data for the patient has not been uploaded and received flow moves to operation 812 where a display indicating that interrogation data is due is generated and the method ends. When a determination is made that interrogation data for the patient has been uploaded and received flow moves to operation 814 where a standard 30-day or 90-day interrogation analysis is performed depending on whether the patient is enrolled in a 30-day or 90-day billing schedule and the method ends.

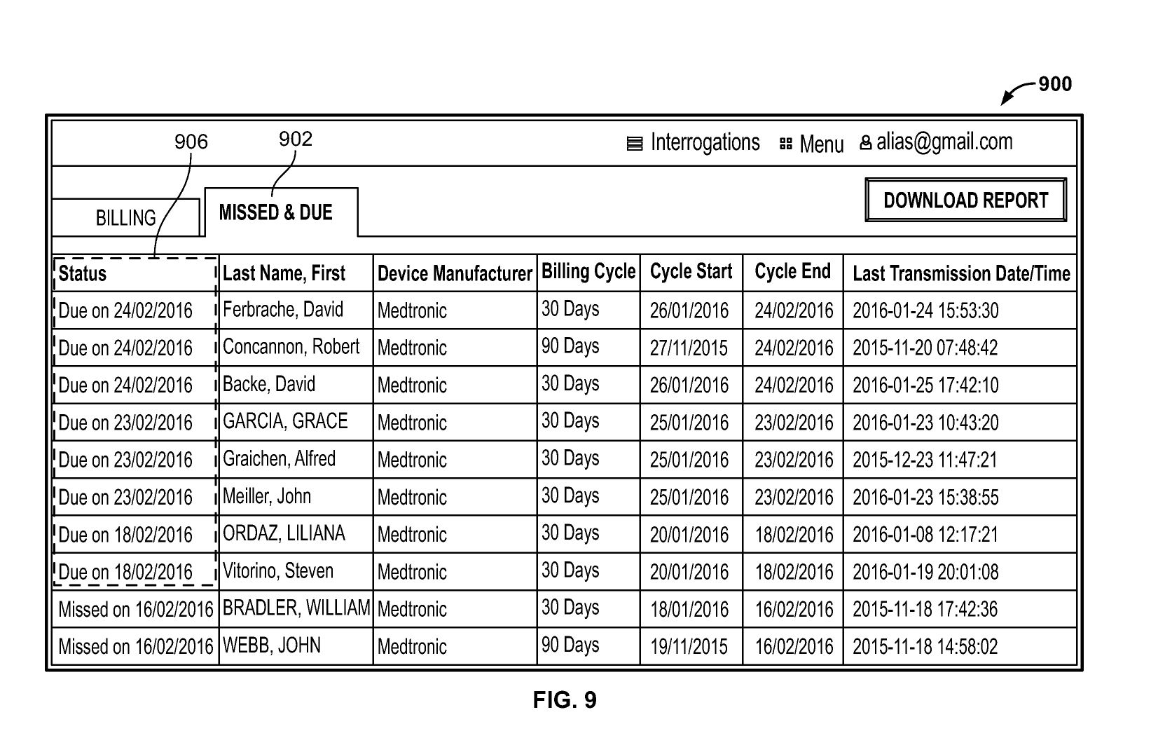

FIG. 9 is a screen shot illustrating an example user interface 900 for determining which patients enrolled in a remote monitoring program are due to upload medical device interrogation data to an interrogation data management system. According to examples a medical care facility administrator or third-party remote monitoring technician may access the missed and due reports user interface by selecting (e.g., by left clicking if using a mouse, or touching a display if using a touch screen) MISSED & DUE 902. Upon making this selection a display may be generated indicating which patients enrolled in a remote monitoring program have a missed status because an interrogation has not been uploaded to an interrogation data management system during a billable term in a previous billing cycle, as well as a due status 904 indicating which patients are due to upload interrogation data to the interrogation data management system.

Graphical user interface 900 may include additional information relevant to the interrogation data management system, including first and last name of patients, device manufacturer, number of days in a patient's billing cycle (e.g., 30-day or 90-day), the start date of a billing cycle in which a missed or due report occurred, an end date for a billing cycle in which a missed or due report occurred, and the date and time of the last interrogation data transmission to the interrogation data management system.

According to aspects a medical care facility administrator or third-party remote monitoring technician may access, via a remote monitoring application, a patient profile for a patient being remotely monitored and input a number of days prior to the end of a billing cycle that they would like to receive a due report if a remotely monitored patient fails to upload billable interrogation data. A medical care facility administrator or third-party remote monitoring technician may determine, for example, that all remotely monitored patients that are enrolled in 30-day billing schedule should have a due report threshold of 10-days preceding the end of a billing cycle, and that all remotely monitored patients that are enrolled in a 90-day billing schedule should have a due report threshold of 30-days preceding the end of a billing cycle. In this way individual medical care facilities may set personalized due report thresholds which may be utilized by the medical care facility, its employees, and contractors, to contact patients that are due to upload interrogation data to an interrogation data management system, which can then be accessed and reviewed by medical care professionals at the medical care facility.

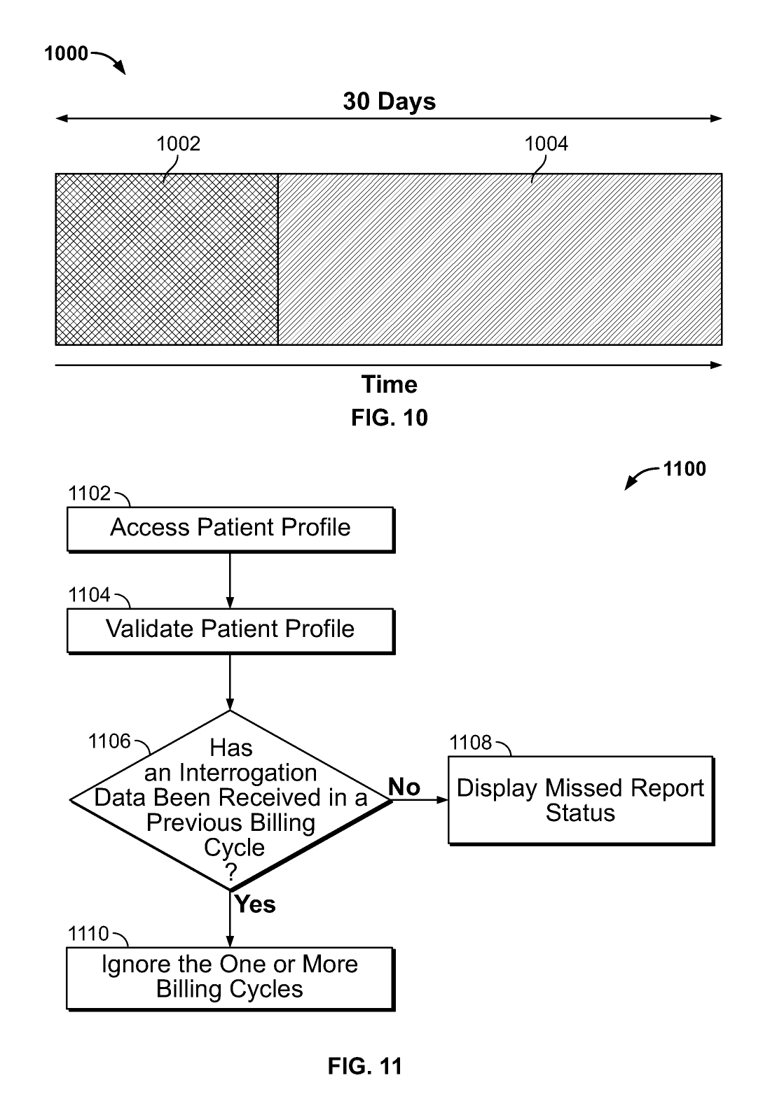

Turning to FIG. 10 a timeline 1000 representative of a single 30-day billing cycle, including 10-day black-out term 1002 which corresponds to days 1-10 at the start of the 30-day billing cycle, and a 20-day billable term 1004 running from the end of the black-out term 1002 to the end of the billing cycle (days 11-30) is provided. The timeline 1000 depicts a billing cycle in which no billable or interim interrogations have been received by an interrogation data management system, resulting in a missed billing cycle for a medical care facility. According to examples, even if interrogation data was received by the interrogation data management system during the black-out term 1002 (i.e., an interim interrogation) a remote monitoring application according to aspects described herein would still classify the billing cycle as a missed billing cycle because no interrogations were received during the 20-day billable term 1004.

The 30-day billing cycle and its corresponding 10-day black-out term 1002 depicted in timeline 1000 may also be representative of other billing cycles, such as a 90-day billing cycle having a 30-day black-out term as may be designated by one or more insurance carriers for remote monitoring of a particular medical device or a patient medical condition that is being remotely monitored.

FIG. 11 is a flow chart 1100 illustrating an exemplary method for determining whether an interrogation data upload from a patient's medical device was missed during a billing cycle. The method begins at operation 1102 where a patient profile for a patient is accessed and continues to operation 1104 where the patient profile is validated.

Validation of the patient profile may entail determining that the patient is enrolled in a remote monitoring program, and subsequently whether the patient is enrolled in a 30-day billing schedule, a 90-day billing schedule, or both a 30-day billing schedule and a 90-day billing schedule.

Moving to operation 1006 a determination is made as to whether interrogation data has been received by an interrogation data management system in the previous billing cycle. When a determination is made that interrogation data has not been received by an interrogation data management system in the previous billing cycle flow moves to operation 1108 where a remote monitoring application may cause a missed report status to be displayed on a graphical user interface. When a determination is made that interrogation has been received by an interrogation data management system in the previous billing cycle flow moves to operation 1110 where a remote monitoring application ignores the one or more billing cycles if queried for a missed report status.

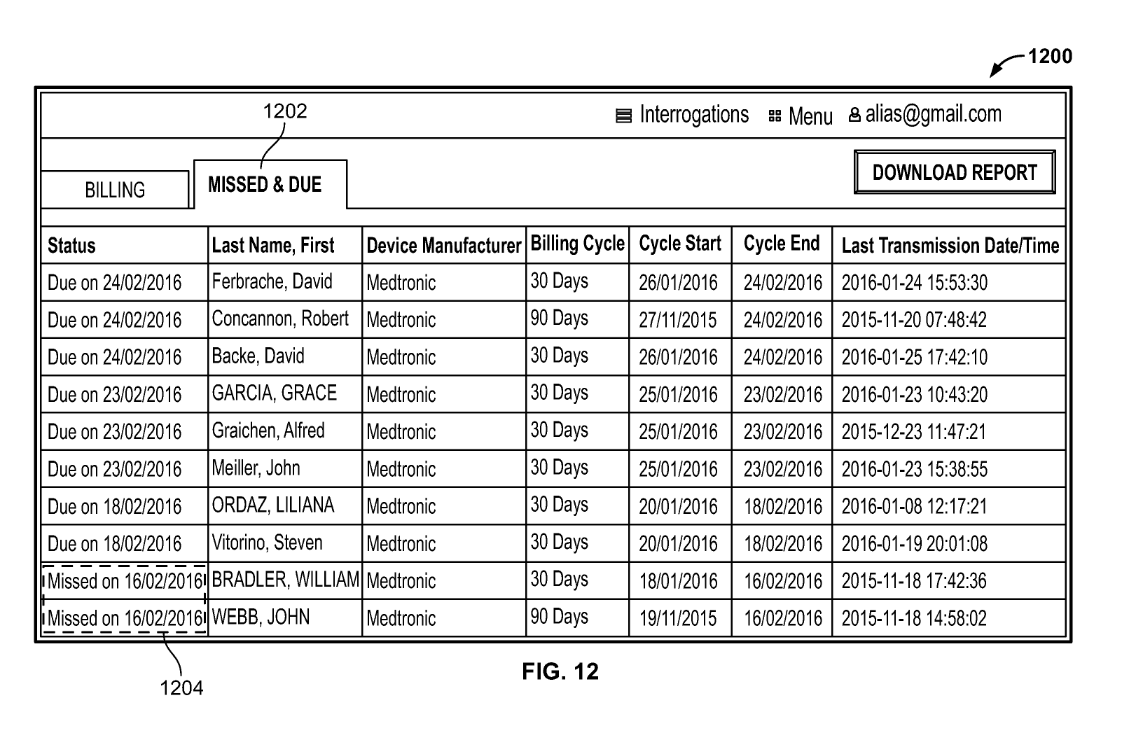

FIG. 12 is a screen shot illustrating an example user interface 1200 for determining which patients enrolled in a remote monitoring program have not uploaded a billable interrogation to an interrogation data management system in a previous billing cycle, thereby missing the bill cycle. According to examples a medical care facility administrator or third-party remote monitoring technician may access the missed and due reports user interface by selecting (e.g., by left clicking if using a mouse, or touching a display if using a touch screen) MISSED & DUE 1202. Upon making this selection a display may be generated indicating which patients enrolled in a remote monitoring program have a missed status 1204 because an interrogation was not uploaded to an interrogation data management system during a billable term in the previous billing cycle, as well as a due status indicating which patients are due to upload interrogation data to the interrogation data management system.

User interface 1200 may include additional information relevant to the interrogation data management system, including first and last name of patients, device manufacturer, number of days in a patient's billing cycle (e.g., 30-day or 90-day), the starting date in a billing cycle in which a missed or due report occurred, the ending date in a billing cycle in which a missed or due report occurred, and the date and time of the last interrogation data transmission to the interrogation data management system.

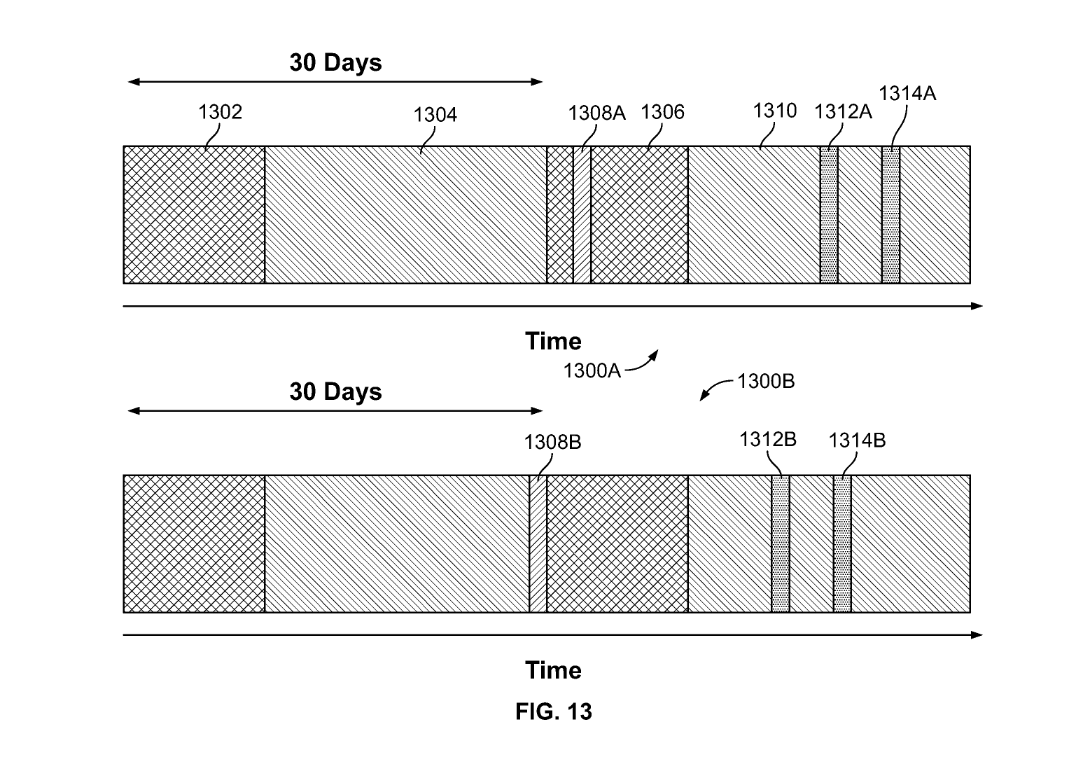

FIG. 13 illustrates a first timeline 1300A and a second timeline 1300B, each of which represent the same two consecutive 30-day billing cycles for a patient. The first timeline 1300A includes a first billing cycle having a 10-day black-out term 1302 followed by a 20-day billable term 1304. No interrogation data was received by an interrogation data management system in the first billing cycle.

The first timeline 1300A also includes a second billing cycle having a 10-day black-out term 1306 followed by a 20-day billable term 1310. Interrogation data from a patient's medical device was received by an interrogation data management system on a first instance 1308A during the black-out term 1306 (i.e., non-billable interim interrogation data), a second instance 1312A during the billable term 1310, and a third instance 1314A during the billable term.

According to examples a remote monitoring application may make a determination that the two consecutive billing cycles may be shifted such that the first instance 1308A interrogation data was received corresponds to the last day of the previous billing cycle as illustrated by the second timeline 1300B, in which the two consecutive billing cycles have been shifted, or calibrated, such that the first instance 1308A interrogation data was received now corresponds to a billable position (i.e., the last day 1308B) in the previously missed billing cycle, and the second instance 1312A and third instance 1314A now also occupy different positions 1312B and 1314B within the billing cycles because of the billing cycle calibration performed by the remote monitoring application. By calibrating the billing cycles a medical care facility may now bill for review of the first instance 1308A, as well as for review of the second instance 1312A. According to additional aspects the billing cycles may be shifted such that the first instance 1308A interrogation data was received corresponds to any day in a billable term of the previous billing cycle.

The 30-day billing cycles depicted in timelines 1300A and 1300B and their corresponding 10-day black-out terms may also be representative of other billing cycles, such as 90-day billing cycles having a 30-day black-out terms as may be designated by one or more insurance carriers for remote monitoring of a particular medical device or a patient medical condition that is being remotely monitored.

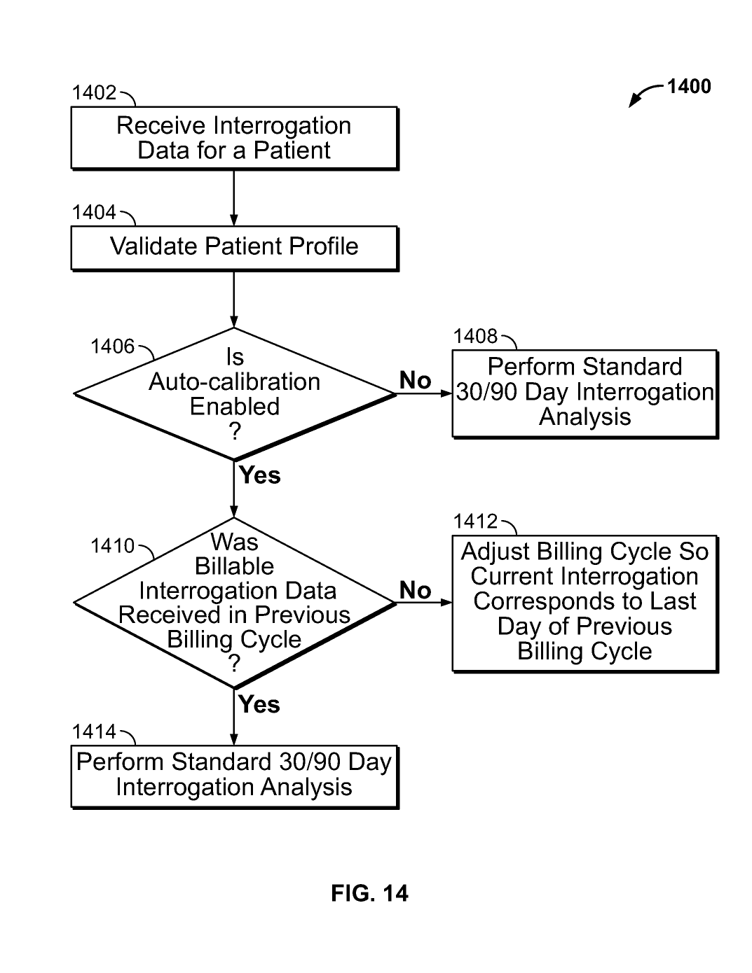

FIG. 14 is a flow chart 1400 illustrating an example method for calibrating a first billing cycle in which an interrogation has been received and a preceding billing cycle in which no billable interrogations were received. The method begins at operation 1402 where interrogation data for a patient is received during a black-out period of the first billing cycle. By way of example the black-out period may encompass the first 10-days of the first billing cycle if the patient is enrolled in a 30-day billing cycle or the first 30-days of the first billing cycle if the patient is enrolled in a 90-day billing cycle. Flow then moves to operation 1404 where a patient profile for the patient is validated.

Validation of the patient profile may entail determining that the patient is enrolled in a remote monitoring program, and subsequently whether the patient is enrolled in a 30-day billing schedule, a 90-day billing schedule, or both a 30-day billing schedule and a 90-day billing schedule. Flow then continues to operation 1406.

At operation 1406 a determination is made as to whether auto-calibration is enabled for the patient. If a determination is made that auto-calibration is not enabled for the patient flow continues to operation 1408 where standard 30-day and/or 90-day interrogation analysis is performed by a remote monitoring application and the method ends. If a determination is made that auto-calibration is enabled for the patient flow moves to operation 1410.

At operation 1410 a determination is made as to whether billable interrogation data from the patient was received by an interrogation data management system in the preceding billing cycle. If a determination is made that no billable interrogation data from the patient was received by an interrogation data management system in the preceding billing cycle flow continues to operation 1412 where the first billing cycle and the preceding billing cycle are adjusted such that the day within the black-out term in which the interrogation data was received by an interrogation data management system corresponds to the last day of the previous billing cycle.

According to other aspects the first billing cycle and the preceding billing cycle may be adjusted such that the day within the black-out term in which the interrogation data was received by an interrogation data management system corresponds to any day within a billable term of the previous billing cycle.

If a determination is made that billable interrogation data from the patient was received by an interrogation data management system in the preceding billing cycle flow moves to operation 1414 where standard 30-day and/or 90-day interrogation analysis is performed by a remote monitoring application and the method ends.

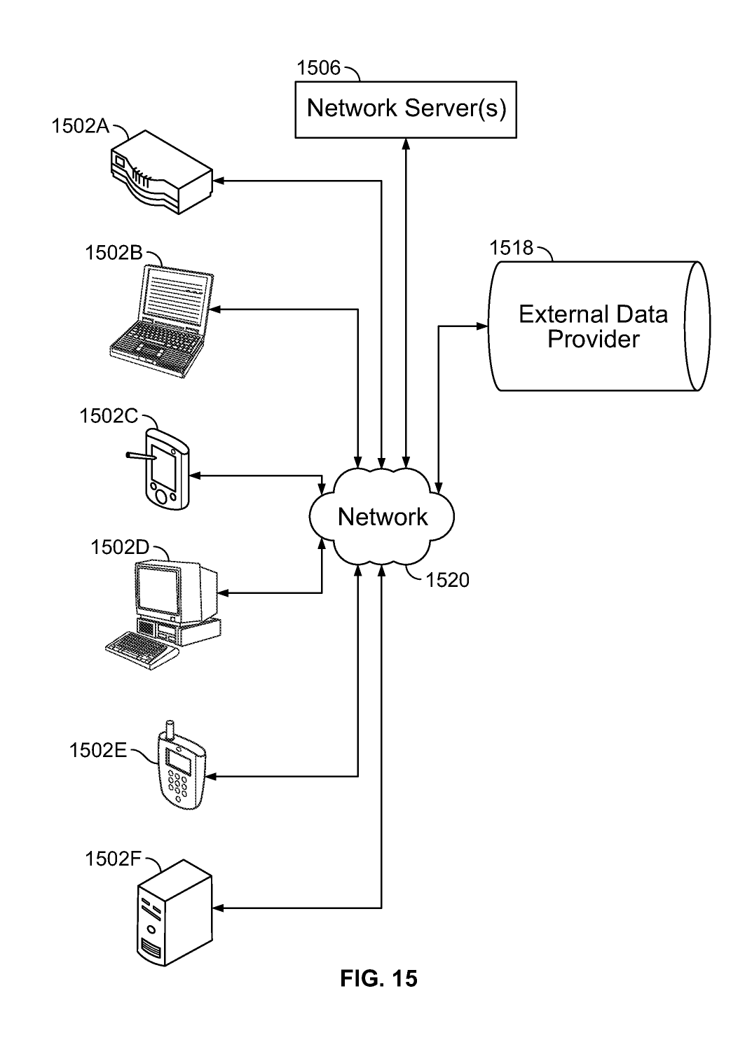

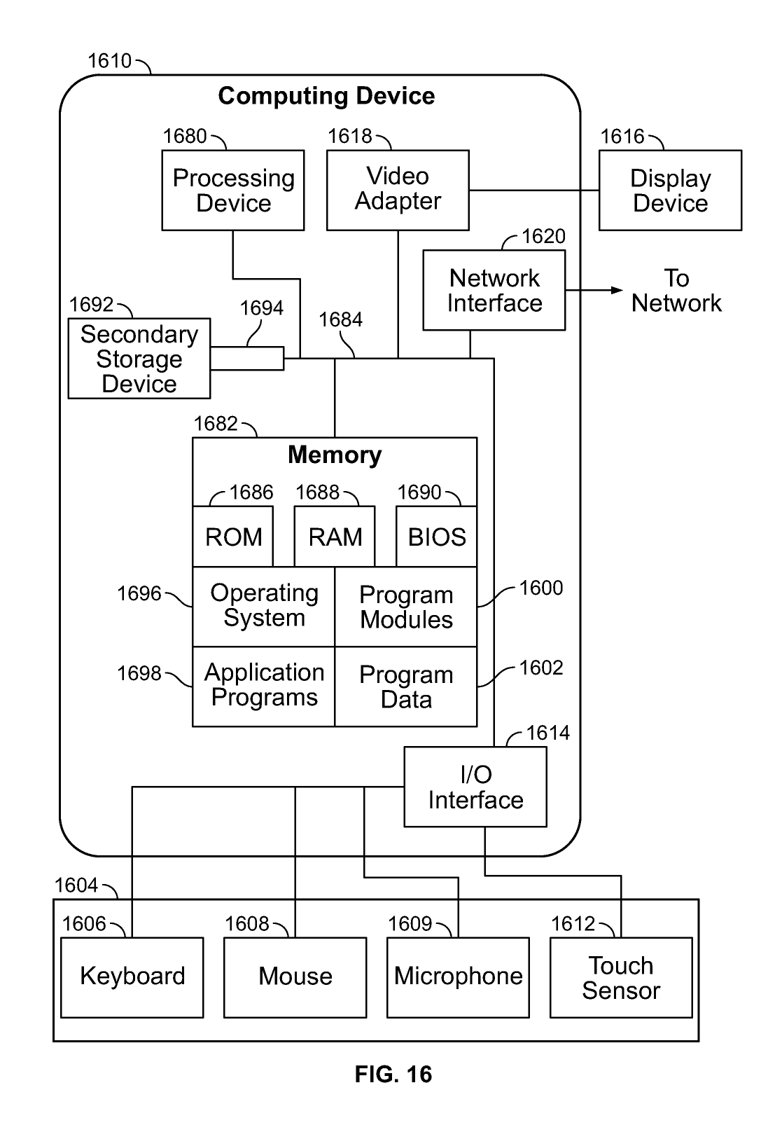

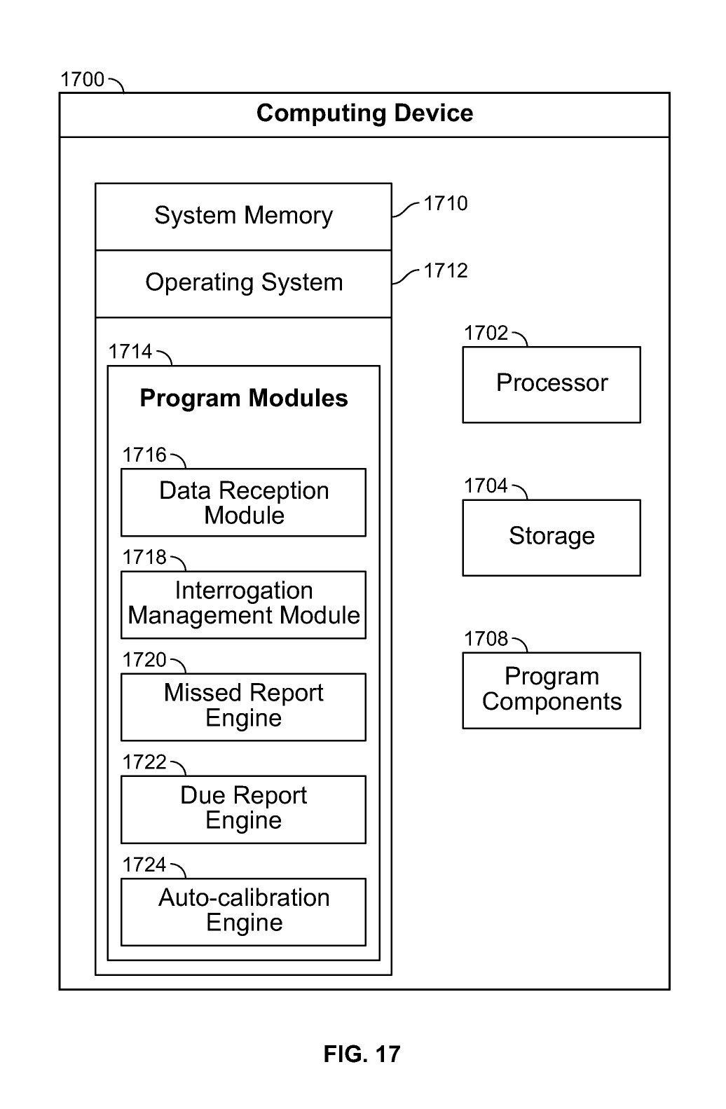

FIG. 15 is a simplified diagram of a distributed computing system in which aspects of the present disclosure may be practiced. According to examples, any of computing devices 1502A (a modem), 1502B (a laptop computer), 1504C (a tablet), 1502D (a personal computer), 1502E (a smart phone), and 1502F (a server) may contain modules, components, engines, etc. for managing remote monitoring interrogation data, including determining the billable status of medical device interrogation data, generating missed and due reports for one or more remotely monitored patients and automatically calibrating patient billing cycles, for example. Additionally, according to aspects discussed herein, any of computing devices 1502A-F may contain necessary hardware for implementing the invention such as described below with regard to FIG. 16 and FIG. 17. Any and all of the remote monitoring functions described herein may be performed, by way of example, at network servers 1506 and/or server 1502 F when computing devices 1502A-F request or receive data from external data provider 1518 by way of network 1520.

FIG. 16 illustrates one aspect in which an exemplary architecture of a computing device according to the disclosure that can be used to implement aspects of the present invention, including any of the plurality of computing devices described herein with reference to the various figures. The computing device illustrated in FIG. 16 can be used to execute the operating system, application programs, and software modules (including the software engines) described herein, for example, with respect to FIG. 17 and program modules 1714, data reception module 1716, interrogation management module 1718, missed report engine 1720, due report engine 1722 and auto-calibration engine 1724. By way of example, the computing device 1610 will be described below as the remote monitoring computing device 1610. To avoid undue repetition, this description of the computing device will not be separately repeated herein for each of the other computing devices, including servers 118 and 114 (depicted in FIG. 1), computing devices 1502A-1502F (depicted in FIG. 15), and computing device 1700 (depicted in FIG. 17) but such devices can also be configured as illustrated and described with reference to FIG. 16.

The computing device 1610 includes, in some embodiments, at least one processing device 1680, such as a central processing unit (CPU). A variety of processing devices are available from a variety of manufacturers, for example, Intel or Advanced Micro Devices. In this example, the computing device 1610 also includes a system memory 1682, and a system bus 1684 that couples various system components including the system memory 1682 to the processing device 1680. The system bus 1684 is one of any number of types of bus structures including a memory bus, or memory controller; a peripheral bus; and a local bus using any of a variety of bus architectures.

Examples of computing devices suitable for the computing device 1610 include a server computer, a desktop computer, a laptop computer, a tablet computer, a mobile computing device (such as a smart phone, an iPod® or iPad® mobile digital device, or other mobile devices), or other devices configured to process digital instructions.

The system memory 1682 includes read only memory 1686 and random access memory 1688. A basic input/output system 1690 containing the basic routines that act to transfer information within computing device 1610, such as during start up, is typically stored in the read only memory 1686.

The computing device 1610 also includes a secondary storage device 1692 in some embodiments, such as a hard disk drive, for storing digital data. The secondary storage device 1692 is connected to the system bus 1684 by a secondary storage interface 1694. The secondary storage devices 1692 and their associated computer readable media provide nonvolatile storage of computer readable instructions (including application programs and program modules), data structures, and other data for the computing device 1610. Details regarding the secondary storage devices 1692 and their associated computer readable media, as well as their associated nonvolatile storage of computer readable instructions (including application programs and program modules) will be more fully described below with reference to FIG. 17.

Although the exemplary environment described herein employs a hard disk drive as a secondary storage device, other types of computer readable storage media are used in other embodiments. Examples of these other types of computer readable storage media include magnetic cassettes, flash memory cards, digital video disks, Bernoulli cartridges, compact disc read only memories, digital versatile disk read only memories, random access memories, or read only memories. Some embodiments include non-transitory media. Additionally, such computer readable storage media can include local storage or cloud-based storage.

A number of program modules can be stored in secondary storage device 1692 or memory 1682, including an operating system 1696, one or more application programs 1698, other program modules 1600 (such as the software engines described herein), and program data 1602. The computing device 1610 can utilize any suitable operating system, such as Microsoft Windows™, Google Chrome™, Apple OS, and any other operating system suitable for a computing device.

In some embodiments, a user provides inputs to the computing device 1610 through one or more input devices 1604. Examples of input devices 1604 include a keyboard 1606, mouse 1608, microphone 1609, and touch sensor 1612 (such as a touchpad or touch sensitive display). Other embodiments include other input devices 1604. The input devices are often connected to the processing device 1680 through an input/output interface 1614 that is coupled to the system bus 1684. These input devices 1604 can be connected by any number of input/output interfaces, such as a parallel port, serial port, game port, or a universal serial bus. Wireless communication between input devices and the interface 1614 is possible as well, and includes infrared, BLUETOOTH® wireless technology, cellular and other radio frequency communication systems in some possible embodiments.

In this example embodiment, a display device 1616, such as a monitor, liquid crystal display device, projector, or touch sensitive display device, is also connected to the system bus 1684 via an interface, such as a video adapter 1618. In addition to the display device 1616, the computing device 1610 can include various other peripheral devices (not shown), such as speakers or a printer.

When used in a local area networking environment or a wide area networking environment (such as the Internet), the computing device 1610 is typically connected to the network through a network interface 1620, such as an Ethernet interface. Other possible embodiments use other communication devices. For example, some embodiments of the computing device 1610 include a modem for communicating across the network.

The computing device 1610 typically includes at least some form of computer readable media. Computer readable media includes any available media that can be accessed by the computing device 1610. By way of example, computer readable media include computer readable storage media and computer readable communication media.

Computer readable storage media includes volatile and nonvolatile, removable and non-removable media implemented in any device configured to store information such as computer readable instructions, data structures, program modules or other data. Computer readable storage media includes, but is not limited to, random access memory, read only memory, electrically erasable programmable read only memory, flash memory or other memory technology, compact disc read only memory, digital versatile disks or other optical storage, magnetic cassettes, magnetic tape, magnetic disk storage or other magnetic storage devices, or any other medium that can be used to store the desired information and that can be accessed by the computing device 1610. Computer readable storage media does not include computer readable communication media.

Computer readable communication media typically embodies computer readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer readable communication media includes wired media such as a wired network or direct-wired connection, and wireless media such as acoustic, radio frequency, infrared, and other wireless media. Combinations of any of the above are also included within the scope of computer readable media.

The computing device illustrated in FIG. 16 is also an example of programmable electronics, which may include one or more such computing devices, and when multiple computing devices are included, such computing devices can be coupled together with a suitable data communication network so as to collectively perform the various functions, methods, or operations disclosed herein.

FIG. 17 is a block diagram illustrating additional physical components (e.g., hardware) of a computing device 1700 with which certain aspects of the disclosure may be practiced. The computing device components described below may have computer executable instructions for receiving interrogation data from a medical device during a current billing cycle; processing data from a patient profile; determining whether a patient's billing schedule corresponds to a first billing schedule or a second billing schedule; determining whether interrogation data from the medical device has been uploaded from the medical device within a billing cycle preceding the current billing cycle; and upon determining that interrogation data from the medical device has not been uploaded from the medical device within the billing cycle preceding the current billing cycle, adjusting the patient's billing schedule such that a date on which interrogation data was received during the current billing cycle corresponds to the last day of the billing cycle preceding the current billing cycle.

In addition, the computing device components described below may have computer executable instructions for accessing a patient profile for a patient having a medical device; determining whether a patient's billing schedule corresponds to a first billing schedule or a second billing schedule; determining whether the patient profile has a due report threshold activated, the due report threshold immediately preceding the end of a current billing cycle; determining whether interrogation data from the medical device has been received in a reimbursable period within a current billing schedule; and upon determining that interrogation data from the medical device has not be received during a reimbursable period within the current billing schedule, displaying, via a graphical user interface, an indication that interrogation data from the medical device is due.

The computing device components described below may also have computer executable instructions for accessing a patient profile for a patient having a medical device; determining whether a patient's billing schedule corresponds to a first billing schedule or a second billing schedule; analyzing one or more billing cycles for the patient to determine whether an interrogation was received for each of the one or more billing cycles; and displaying, via a graphical user interface, an indication of whether an interrogation was received for each of the one or more billing cycles.

According to another aspect the computing device components described below may have computer executable instructions for accessing a patient profile for a patient having a medical device; determining whether a patient's billing schedule corresponds to a first billing schedule or a second billing schedule; analyzing interrogation data for a billing cycle received from the medical device, the interrogation data corresponding to a first interrogation; determining whether the interrogation data falls within a black-out term corresponding to the first billing schedule or the second billing schedule; determining whether interrogation data corresponding to a second interrogation within the cycle has been received, the second interrogation preceding the first interrogation; and displaying, via a graphical user interface, an indication of whether the first interrogation is a reimbursable interrogation.

Computing device 1700 may perform these functions alone or in combination with a distributed computing network such as those described with regard to FIG. 15 which may be in operative contact with a personal computing device, a tablet computing device and/or mobile computing device which may communicate and process the one or more of the program modules described in FIG. 17 including data reception module 1716, interrogation management module 1718, missed report engine 1720, due report engine 1722 and auto-calibration engine 1724. According to additional examples, computing device 1700 may be in communicative contact via the distributed computing network described in FIG. 15 and computing device 1700 may describe any of devices 1502A, 1502B, 1502C, 902D, 1502E and 1502F. Additionally, computing device 1700 may represent computing devices 106, 114, 118, 126, 128 and 136 with regard to the descriptions provided for FIG. 1 and/or computing device 1610 as described above with regard to FIG. 16.

In a basic configuration, the computing device 1700 may include at least one processor 1702 and a system memory 1710. Depending on the configuration and type of computing device, the system memory 1710 may comprise, but is not limited to, volatile storage (e.g., random access memory), non-volatile storage (e.g., read-only memory), flash memory, or any combination of such memories. The system memory 1710 may include an operating system 1712 and one or more program modules 1714 suitable for performing the functions described herein with regard to management of remote monitoring interrogation data, such as one or more components in regards to FIG. 17 and, in particular, data reception module 1716, interrogation management module 1718, missed report engine 1720, due report engine 1722 and auto-calibration engine 1724. The operating system 1712, for example, may be suitable for controlling the operation of the computing device 1700. Furthermore, embodiments of the disclosure may be practiced in conjunction with a graphics library, other operating systems, or any other application program and are not limited to any particular application or system.

The computing device 1700 may have additional features or functionality. For example, the computing device 1700 may also include additional data storage device (removable and/or non-removable) such as, for example, magnetic disks, optical disks, or tape. Such additional storage is illustrated in FIG. 17 by storage 1704. Storage may also occur via the distributed computing networks described in FIG. 1 and FIG. 15. For example, computing device 1700 may communicate via network 1520 in FIG. 15 and data may be stored within network servers 1506 and transmitted back to computing device 1700 via network 1520 if it is determined that such stored data is necessary to execute one or more functions described herein.. Additionally, computing device 1700 may communicate via network 108 and network 120 in FIG. 1 and data may be stored within servers 106, 114 and 118 and transmitted back to computing device 1700 via network 108 and network 120 if it is determined that such stored data is necessary to execute one or more functions described herein.

As stated above, a number of program modules and data files may be stored in the system memory 1710. While executing the processor 1702, the program modules 1714 (e.g., data reception module) may perform processes including, but not limited to, the aspects described herein. Other program modules that may be used in accordance with aspects of the present disclosure, and in particular may include a medical device communication engine, a billing code optimization engine, and a HIPPA compliance engine, for example.

The various embodiments described above are provided by way of illustration only and should not be construed to limit the claims attached hereto. Those skilled in the art will readily recognize various modifications and changes that may be made without following the example embodiments and applications illustrated and described herein, and without departing from the true spirit and scope of the following claims.

1-21. (canceled)

22. A computer-implemented method for calibrating medical device interrogation billing cycles, comprising:
receiving, on a first specific date, first interrogation data from a medical device during an unbillable time period of a current billing cycle;
determining whether previous interrogation data from the medical device had been received during a billable time period of a preceding billing cycle; and
in response to determining that previous interrogation data from the medical device had not been received during the billable time period of the preceding billing cycle, shifting both the preceding billing cycle and the current billing cycle backward in time enabling the first specific date of the first interrogation data to fall within a billable time period of the shifted preceding billing cycle.

23. The computer-implemented method of claim 22, further comprising
receiving, on a second specific date, second interrogation data from the medical device during a billable period of time of the current billing cycle,
wherein the shifting of both the preceding billing cycle and the current billing cycle backward in time further enables the second specific date of the second interrogation data to fall within a billable time period of the shifted current billing cycle; and
billing for both the first interrogation data falling within the billable time period of the shifted preceding billing cycle and the second interrogation data falling within the billable time period of the shifted current billing cycle.

24. The computer-implemented method of claim 23, further comprising,
in response to determining that the previous interrogation data from the medical device had been received during the billable time period of the preceding billing cycle, billing for only the second interrogation data.

25. The computer-implemented method of claim 22, wherein shifting both the preceding cycle and the current billing cycle backward in time enabling the first specific date of the first interrogation data to fall within the billable time period of the shifted preceding billing cycle includes enabling the first specific date of the first interrogation data to fall on a last day of the of the billable time period of the shifted preceding billing cycle.

26. The computer-implemented method of claim 22, further comprising determining a length of time of at least one of the preceding billing cycle and the current billing cycle.

27. The computer-implemented method of claim 26, wherein the determined length of time is a length of 30 days or 90 days.

28. The computer-implemented method of claim 26, wherein shifting both the preceding billing cycle and the current billing cycle backward in time includes maintaining the determined length of time of the at least one of the preceding billing cycle and the current billing cycle.

29. The computer-implemented method of claim 26, wherein the first interrogation data includes a patient identifier and wherein determining the length of time of the at least one of the preceding billing cycle and the current billing cycle is based on the patient identifier.

30. The computer-implemented method of claim 22, wherein the medical device comprises an implantable medical device.

31. A computer-implemented method for calibrating medical device interrogation billing cycles, comprising:
receiving first interrogation device data on a specific date from a medical device during an unbillable period of a current billing cycle, the current billing cycle having a current start date and a current end date, and having a current billable time period between the current start date and the current end date inclusive of the current end date;
determining whether previous interrogation device data from the medical device was received during a preceding billable time period of a preceding billing cycle, the preceding billing cycle immediately preceding the current billing cycle, the preceding billing cycle having a preceding start date and a preceding end date, wherein the preceding billable time period of the preceding billing cycle is between the preceding start date and the preceding end date inclusive of the preceding end date; and
in response to determining that the previous interrogation device data was not received during the preceding billable time period:
resetting the preceding start and end dates of the preceding billing cycle, the preceding billable time period being reset in accordance with the reset preceding start and end dates, the reset preceding start and end dates having been reset so that the specific date of receiving the first interrogation device data falls within the reset preceding billable time period; and
resetting the current start and end dates of the current billing cycle, the current billable time period being reset in accordance with the reset current start and end dates, the current start and end dates reset so that the reset current start date is immediately subsequent the reset preceding end date.

32. The computer-implemented method of claim 31, further comprising,
receiving, on a second specific date, second interrogation device data from the medical device during the current billable time period of the current billing cycle,
wherein resetting both the preceding start and end dates and the current start and end dates, enables the second specific date of the second interrogation device data to fall within the reset current billable time period of the reset current billing cycle; and
billing for both the first interrogation device data falling within the reset preceding billable time period of the reset preceding billing cycle and for the second interrogation device data falling within the reset current billable time period of the reset current billing cycle.

33. The computer-implemented method of claim 32, further comprising,
in response to determining that the previous interrogation device data was not received during the preceding billable time period,
billing for only the second interrogation device data.

34. The computer-implemented method of claim 31, wherein resetting the preceding start and end dates of the preceding billing cycle enables the specific date of receiving the first interrogation device data to fall on the last day of the reset preceding billable time period.

35. The computer-implemented method of claim 31, further comprising determining a length of time of at least one of the preceding billing cycle and the current billing cycle.

36. The computer-implemented method of claim 35, wherein the determined length is a length of 30 days or 90 days.

37. The computer-implemented method of claim 35, wherein resetting the preceding start and end dates and resetting the current starting and end dates includes maintaining the determined length of the at least one of the preceding billing cycle and the current billing cycle.

38. The computer-implemented method of claim 35, wherein the first interrogation data includes a patient identifier and wherein the determining the length of at least one of the preceding billing cycle and the current billing cycle is based on the patient identifier.

39. The computer-implemented method of claim 31, wherein the medical device comprises an implantable medical device.

40. A computer-implemented method for calibrating medical device interrogation billing cycles, comprising:
receiving, on a first specific date, first interrogation data from a medical device during an unbillable time period of a current billing cycle;
receiving, on a second specific date, second interrogation data from the medical device during a billable period of time of the current billing cycle;
accessing a patient profile based on one or both of the received first interrogation data and the received second interrogation data;
validating the patient profile to determine a billing schedule associated with the patient;
determining whether previous interrogation data from the medical device had been received during a billable time period of a preceding billing cycle;
in response to determining that previous interrogation data from the medical device had not been received during the billable time period of the preceding billing cycle,
shifting both the preceding billing cycle and the current billing cycle backward in time enabling the first specific date of the first interrogation data to fall within a billable time period of the shifted preceding billing cycle and enabling the second interrogation data to fall within a billable time period of the shifted current billing cycle; and
billing for both the first interrogation data falling within the billable time period of the shifted preceding billing cycle and the second interrogation data falling within the billable time period of the shifted current billing cycle; and
in response to determining that the previous interrogation data from the medical device had been received during the billable time period of the preceding billing cycle, billing for only the second interrogation data.

41. The computer-implemented method of claim 40, wherein the medical device comprises an implantable medical device.
